(19) United States
(12) Patent Application Publication
(10) Pub. No.: US 2023/0053173 A1
(43) Pub. Date: Feb. 16, 2023

(54) INTERFACE BETWEEN A SURGICAL ROBOT ARM AND A ROBOTIC SURGICAL INSTRUMENT

(71) Applicant: CMR SURGICAL LIMITED, Canbridge (GB)

(72) Inventors: Kenneth Focht, Needham, MA (US); Joseph Gordon, Mansfield, MA (US); Peter Calvin Costello, Raynham, MA (US); Daniel P. Smith, Portsmouth, RI (US); Rene Raymond Robert, East Greenwich, RI (US)

(21) Appl. No.: 17/646,158

(22) Filed: Dec. 28, 2021

Related U.S. Application Data
(63) Continuation-in-part of application No. 17/400,788, filed on Aug. 12, 2021.

(51) Int. Cl. A61B 34/30

(52) U.S. Cl. CPC A61B 34/30; A61B 2034/302; A61B 34/37

(57) ABSTRACT

A drive unit for a surgical robot arm, the robot arm being configured to engage a robotic surgical instrument, the drive unit comprising a plurality of drive interface elements, each drive interface element having a longitudinal axis; a plurality of actuators configured to drive the plurality of drive interface elements, each actuator of the plurality of actuators being configured to drive one of the plurality of drive interface elements so as to cause that drive interface element to be displaced along its longitudinal axis in a first direction,

wherein the drive unit is configured such that, when the surgical robot arm engages the robotic surgical instrument, the longitudinal axis of each drive interface element is aligned with a longitudinal axis of a respective instrument interface element in the instrument and each drive interface element is configured such that the displacement of said drive interface element along its longitudinal axis in the first direction causes a displacement of the respective instrument interface element along its longitudinal axis in the first direction.

### CROSS-REFERENCE TO RELATED APPLICATION

This application is a continuation-in-part of U.S. patent application Ser. No. 17/400,788 filed on Aug. 12, 2021 which is hereby incorporated herein by reference in its entirety for all purposes.

### BACKGROUND

It is known to use robots for assisting and performing surgery. FIGS. 1 and 2 show a typical surgical robot 100 which comprises a base 101, an arm 102 and an instrument 103. The base supports the robot, and is itself attached rigidly to, for example, the operating theatre floor, the operating theatre ceiling or a trolley. The arm 102 extends between the base and the instrument. The arm is formed of multiple links 105 and is articulated by means of multiple joints 104 between the links which are used to position the surgical instrument 103 in a desired location and orientation relative to the patient. The surgical instrument is attached to the distal end of the robot arm. As seen in FIG. 2, the link of the robot arm which connects to the instrument is the terminal link 205. During an operation, the surgical instrument can penetrate the body of a patient at a port so as to access the surgical site.

A typical surgical instrument 103 as shown in FIG. 3 comprises an instrument interface 301 by means of which the surgical instrument connects to the terminal link 205 of the robot arm 102. A shaft 302 extends between the instrument interface 301 and an articulation 303. The articulation terminates in an end effector 304 and permits the end effector to move relative to the shaft 302.

It is desirable to develop a surgical robot able to control an attachable surgical instrument such that the end effector of the surgical instrument can be positioned in the desired location and orientation relative to a patient and be actuated so as to perform the desired surgical procedure. It is also desirable that the means for controlling the instrument is compact and light and that force can be transferred from the robot arm to the instrument in an efficient manner. Finally, it is desirable that the surgical instrument can be easily attached and detached from the robot arm.

### SUMMARY OF INVENTION

According to a first embodiment, there is provided a drive unit for a surgical robot arm, the robot arm being configured to engage a robotic surgical instrument, the drive unit comprising: a plurality of drive interface elements, each drive interface element having a longitudinal axis; and a plurality of actuators configured to drive the plurality of drive interface elements, each actuator of the plurality of actuators being configured to drive one of the plurality of drive interface elements so as to cause that drive interface element to be displaced along its longitudinal axis in a first direction, wherein the drive unit is configured such that, when the surgical robot arm engages the robotic surgical instrument, the longitudinal axis of each drive interface element is aligned with a longitudinal axis of a respective instrument interface element in the instrument and each drive interface element is configured such that the displacement of said drive interface element along its longitudinal axis in the first direction causes a displacement of the respective instrument interface element along its longitudinal axis in the first direction.

The drive unit may be configured such that, when the surgical robot arm engages the robotic surgical instrument, the longitudinal axis of each drive interface element is collinear with the longitudinal axis of the respective instrument interface element in the instrument.

Each drive interface element may have a proximal end and a distal end and the first direction may extend from the proximal end to the distal end.

The drive unit may be configured such that, when the surgical robot arm engages the robotic surgical instrument, the plurality of drive interface elements are not secured to the respective instrument interface elements in the instrument.

The drive unit may comprise a load cell unit configured to sense a load applied to one or more of the plurality of drive interface elements by the plurality of actuators.

The drive unit may be configured such that, when the surgical robot arm engages the robotic surgical instrument, the load cell unit is positioned between the plurality of drive interface elements and the respective instrument interface elements in the instrument.

The load cell unit may comprise a plurality of pads, and the drive unit may be configured such that, when the surgical robot arm engages the robotic surgical instrument, each of the pads is positioned such that the load cell unit transfers the displacement of each drive interface element along its longitudinal axis in the first direction to a displacement of the respective instrument interface element along its longitudinal axis in the first direction.

Each actuator of the plurality of actuators may be configured to drive a drive interface element so as to cause that drive interface element to be displaced along its longitudinal axis in the first direction only.

Each actuator of the plurality of actuators may be configured to drive a drive interface element so as to cause that drive interface element to be displaced along its longitudinal axis in the first direction and in a second direction, wherein the second direction is opposite to the first direction.

The drive unit may be configured such that, when the surgical robot arm engages the robotic surgical instrument, the displacement of a drive interface element along its longitudinal axis in the second direction does not cause a displacement of the respective instrument interface element in the instrument.

The drive unit may be configured such that, when the surgical robot arm engages the robotic surgical instrument, the displacement of a drive interface element along its longitudinal axis in the second direction causes a displacement of the respective instrument interface element along its longitudinal axis in the second direction.

The drive unit may have only four drive interface elements.

The drive unit may comprise a housing and a key positioned between a drive interface element and the housing, wherein the key is secured to the housing and is configured to engage the respective drive interface element such that the drive interface element can be displaced relative to the key in a direction along the longitudinal axis of the drive interface element.

The drive interface element may comprise a slot, the slot being parallel to the longitudinal axis of the drive interface element, and the key may be configured to slide within the slot when the drive interface element is displaced along its longitudinal axis.

According to a second embodiment, there is provided a robotic surgical instrument configured to engage a surgical robot arm, the instrument comprising: a plurality of instrument interface elements, each instrument interface element having a longitudinal axis, the instrument being configured such that, when the instrument engages the surgical robot arm, the longitudinal axis of each instrument interface element is aligned with a longitudinal axis of a respective drive interface element in the surgical robot arm and each instrument interface element is configured such that a displacement of the respective drive interface element along its longitudinal axis in a first direction causes a displacement of the instrument interface element along its longitudinal axis in the first direction.

The instrument may be configured such that, when the instrument engages the surgical robot arm, the longitudinal axis of each instrument interface element is colinear with the longitudinal axis of the respective drive interface element in the surgical robot arm.

The instrument may be configured such that, when the robotic surgical instrument engages the surgical robot arm, the plurality of instrument interface elements are not secured to the respective drive interface elements.

The instrument may be configured such that, when the instrument engages the surgical robot arm, each instrument interface element is configured to be engaged by a pad of a load cell unit of the surgical robotic arm such that a displacement of the respective pad in the first direction causes a displacement of the instrument interface element along its longitudinal axis in the first direction.

The instrument may be configured such that, when the instrument engages the surgical robot arm, the instrument interface elements are not secured to the respective pads of the load cell unit.

The instrument may comprise: a shaft having a longitudinal axis extending between a proximal end and a distal end, and an articulation and an end effector disposed at the distal end of the shaft, the articulation being configured to articulate the end effector, wherein the plurality of instrument interface elements are disposed at the proximal end of the shaft and the longitudinal axis of each of the instrument interface elements is parallel to the longitudinal axis of the shaft.

The first direction may be from the proximal end to the distal end of the shaft.

The instrument may be configured such that, when the instrument engages the surgical robot arm, each instrument interface is secured to a driving element, such that the instrument is configured to translate drive from each of the driving elements into articulation of the end effector.

The plurality of instrument interface elements may be disposed circumferentially about the longitudinal axis of the shaft.

Each instrument interface element may be configured such that the displacement of the respective drive interface element along its longitudinal axis in a second direction opposite to the first direction does not cause a displacement of the instrument interface element.

Each instrument interface element may be configured such that the displacement of the respective drive interface element along its longitudinal axis in a second direction opposite to the first direction causes a displacement of the instrument interface element along its longitudinal axis in the second direction.

The instrument may have only 4 instrument interface elements.

According to a third embodiment, there is provided a robotic surgical instrument configured to engage a surgical robot arm, the instrument comprising: an articulation configured to articulate an end effector, the articulation driveable by a plurality of driving elements; and a plurality of instrument interface elements, each of the plurality of instrument interface elements having a longitudinal axis and engaging a respective driving element of the plurality of driving elements, wherein the instrument is configured such that, when the instrument engages the surgical robot arm, each of the plurality of instrument interface elements is configured to be displaced along its longitudinal axis in a first direction by a respective drive interface element of the surgical robot arm, and each instrument interface element is configured such that a displacement of the instrument interface element along its longitudinal axis in the first direction causes a displacement of the respective driving element along said longitudinal axis in the first direction.

The robotic surgical instrument may comprise a shaft.

Each instrument interface element may engage a respective pulley about which the respective driving element is at least partially wound.

The instrument may be configured such that a displacement of the instrument interface element along its longitudinal axis in the first direction causes a displacement of the respective driving element relative to its respective pulley.

Each instrument interface element may comprise a groove in which the respective pulley is situated.

Each pulley may have a diameter, wherein a position at one end of the diameter may be aligned with the longitudinal axis of the respective instrument interface element.

A position at the other end of the diameter of the main body of the pulley may be located within a projected profile of the instrument shaft

Each instrument interface element may be secured to its respective pulley.

Each instrument interface element may comprise a toothed rack configured to engage a respective toothed gear about which the respective driving element is at least partially wound.

### BRIEF DESCRIPTION OF THE FIGURES

FIG. 1 shows a surgical robot and a patient.

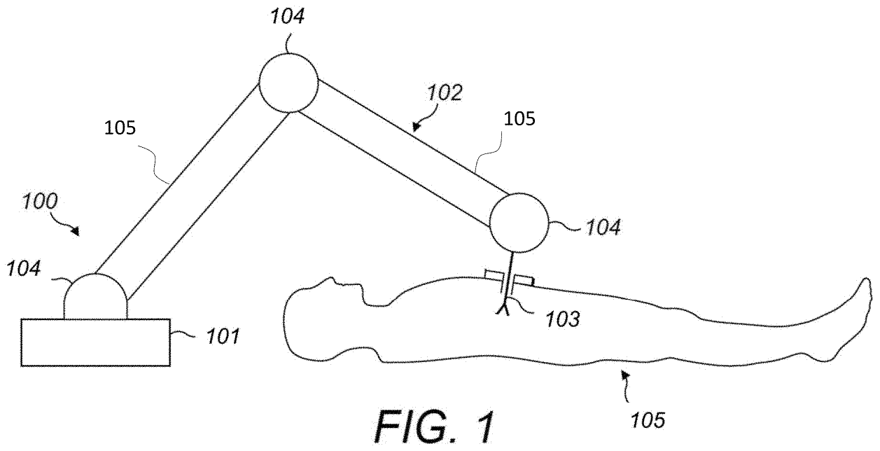

FIG. 2 shows a surgical robot and associated control system.

FIG. 3 shows an instrument.

FIG. 4A shows a drive unit which can be attached to the distal end of a robot arm.

FIG. 4B shows a portion of the drive unit.

FIG. 4C shows a further view of the drive unit.

FIG. 5A shows the proximal end of the instrument including an instrument interface.

FIG. 5B shows the instrument including the instrument shaft and end effector engaged with the robot arm.

FIG. 6A shows the drive unit engaged with the instrument.

FIG. 6B shows a drape cup.

FIG. 6C shows the drive unit engaged with the instrument and additionally the drape cup positioned between the drive unit and the instrument.

FIG. 7 shows the distal end of the instrument including the end effector.

FIG. 8A shows a different view of the drive unit engaged with the instrument.

FIG. 8B shows the instrument interface elements and respective pulleys.

FIG. 8C shows the cross section of a single instrument interface element.

FIG. 9 shows a proximal end of the instrument including an instrument interface and an attached drive unit according to an alternative embodiment.

FIG. 10 shows a different view of the proximal end of the instrument and drive unit according to the alternative embodiment.

### DETAILED DESCRIPTION

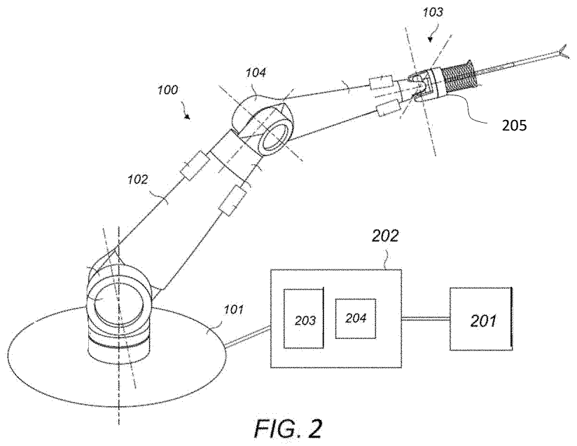

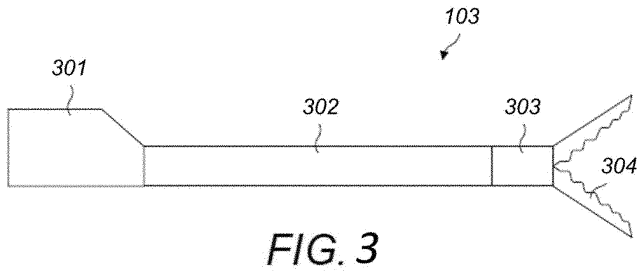

The following describes a robot comprising a robot arm and an instrument. The arm is generally of the form seen in FIG. 2. The instrument is generally of the form seen in FIG. 3. The instrument 103 can be attached to, and detached from, the terminal link 205 of the robot arm 102.

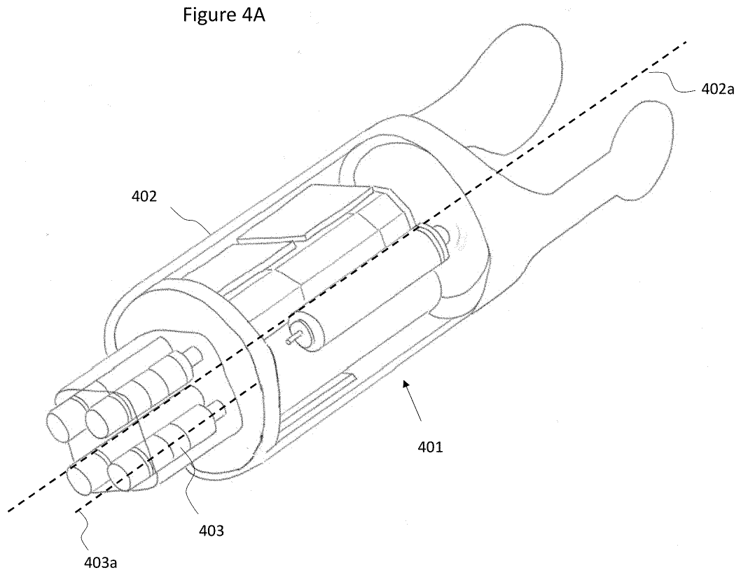

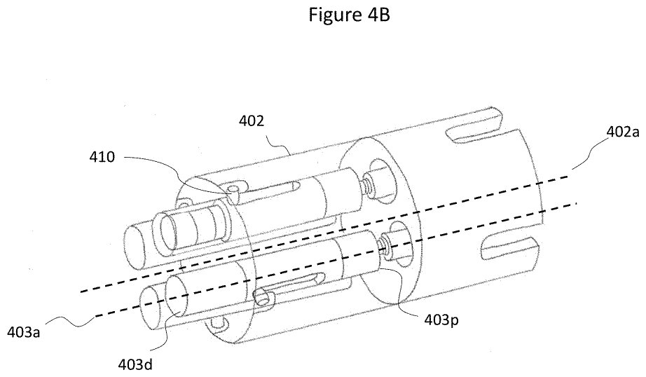

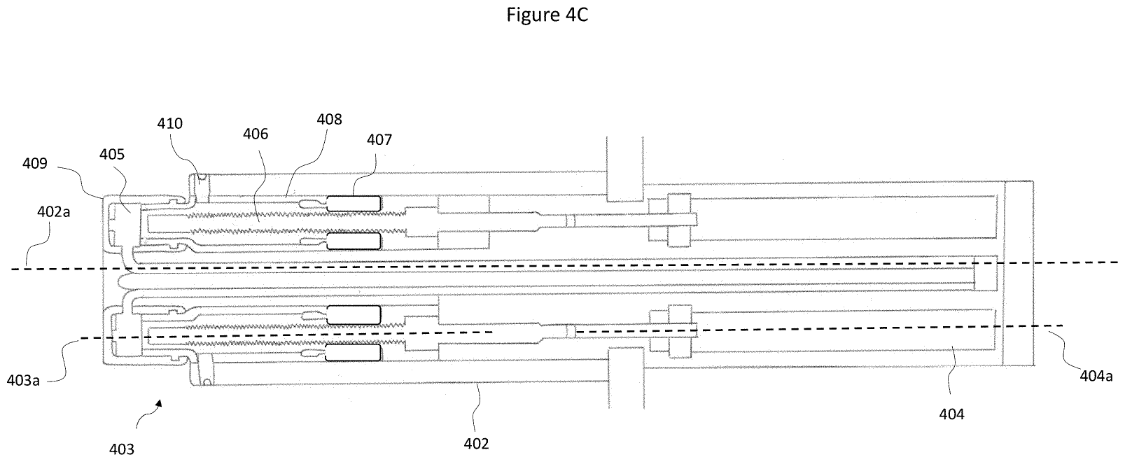

The terminal link 205 of the arm 102 terminates in a drive unit 401, seen in FIG. 4A. The drive unit 401 is located at the distal end of the arm. When the instrument 103 is attached to the arm 102, the drive unit 401 interfaces with the interface 301 of the instrument. As will be explained in more detail below, the drive unit transfers drive from the arm to the instrument in order to cause the articulation 303 to articulate (i.e. move) the end effector 304.

The drive unit 401 comprises a housing 402. In the example seen in FIG. 4A, the housing has a generally cylindrical shape, but may take the form of another shape. The drive unit comprises a plurality of drive interface elements 403. The plurality of drive interface elements are positioned partially within the drive unit housing 402. FIG. 4B shows the portion of the drive unit housing 402 which houses the drive interface elements. In the example seen in FIGS. 4A and 4B, the cylindrical housing 402 has a longitudinal axis 402a. In this example, each drive interface element 403 has the shape of a rod (e.g. a cylindrical rod) and has a proximal end 403p and a distal end 403d, as seen in FIG. 4B. Each drive interface element extends from its proximal end to its distal end in a direction parallel to the longitudinal axis of the housing. Each drive interface element has a longitudinal axis 403a which is parallel to the longitudinal axis 402a of the housing. Each drive interface element 403 protrudes from the housing 402. Each drive interface element protrudes from the housing (e.g. from the distal end of the housing) in a direction parallel to the housing's longitudinal axis.

In the example seen in FIGS. 4A and 4B, the drive unit includes four drive interface elements 403. In other examples the drive unit may include more or fewer drive interface elements. In this example, each drive interface element has the form of a cylindrical rod. However according to other examples, drive interface elements may take any other suitable shape. In the example depicted, each drive interface element has the same shape and size, but in other examples, the drive interface elements may not be of uniform size and/or shape. In the example seen in FIG. 4B, each drive interface element 403 is located within a cylindrical recess in the drive unit housing 402.

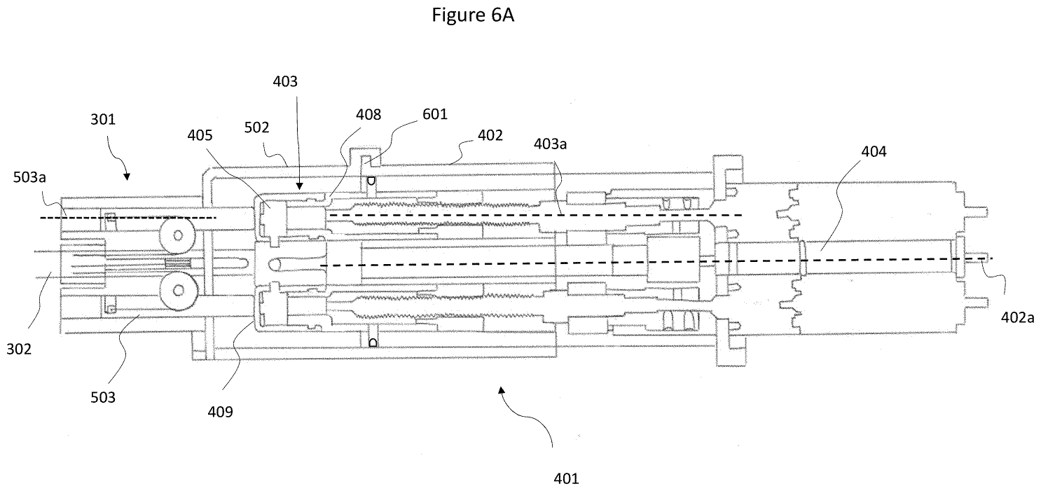

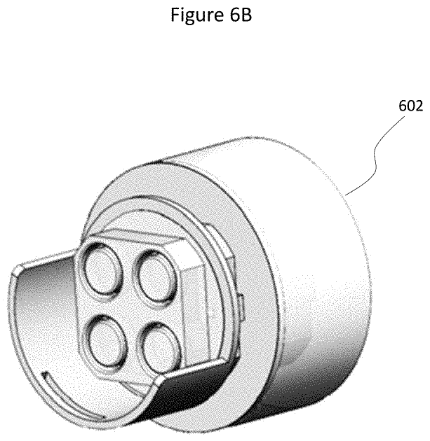

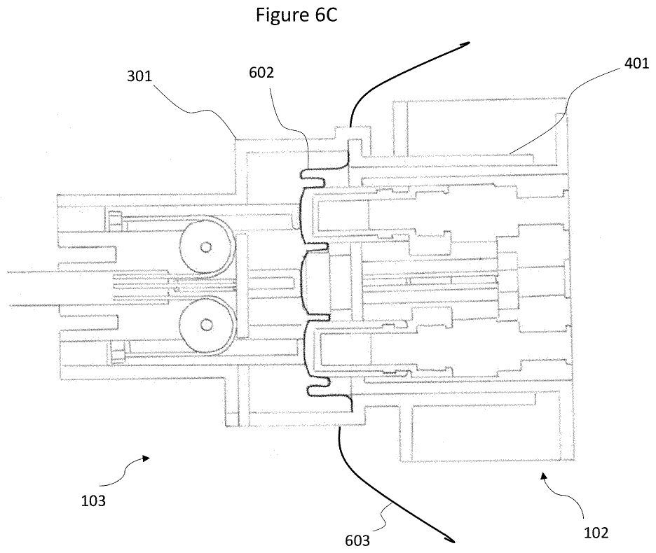

As illustrated in FIG. 4C, the drive unit 401 also comprises a plurality of actuators 404. The plurality of actuators are positioned within drive unit housing 402. The drive unit seen in FIGS. 4A and 4C comprises four actuators (not all shown). In other examples the drive unit may comprise more of fewer actuators. In the present example the drive unit comprises the same number of actuators as the number of drive interface elements. Each actuator is positioned adjacent to a respective drive interface element 403. Each actuator transfers drive to its respective drive interface element. In other examples, such as that shown in FIG. 6A, the drive unit may include an actuator which is configured to transfer drive to more than one drive interface element. Returning to the example shown in FIGS. 4A, 4B and 4C, each drive interface element has a longitudinal axis 403a and each actuator has a longitudinal axis 404a. FIG. 4C illustrates only two of the four drive interface elements and two respective actuators and shows that the longitudinal axis 403a of each of the two drive interface elements is aligned with the longitudinal axis of its respective actuator 404a. In FIG. 4C, the longitudinal axis of each drive interface element is colinear with the longitudinal axis of its respective actuator. The longitudinal axis of each actuator also extends in a direction which is parallel to the longitudinal axis of the housing 402a.

Each drive interface element 403 is driven by a respective actuator 404. Specifically, each actuator causes its respective drive interface element 403 to be displaced. The displacement of a drive interface element 403 is in a direction along the longitudinal axis of the drive interface element 403a. In the drive unit seen in FIGS. 4A, 4B and 4C, each drive interface element 403 can be displaced by its respective actuator along its longitudinal axis only in the direction from its proximal end to its distal end. When the arm attaches to an instrument, this direction is towards the end effector of the instrument. However, in other examples, the drive interface elements may also be displaced by their respective actuators in the opposite direction, from their distal end to their proximal end i.e. away from the end effector of an attached instrument.

As seen in FIG. 2, the surgical robot 100 forms part of a system also including a surgeon command interface 201 and a control unit 202. The control unit comprises a processor 203 and a memory 204. The memory 204 stores in a non-transient way software that is executable by the processor 203 to control the operation of the actuators to cause the arm 102 to move as desired. The software can control the processor 203 to cause the actuators 404 to drive in dependence on inputs from the surgeon command interface. The control unit 202 is coupled to the actuators for driving them in accordance with outputs generated by execution of the software. The surgeon command interface 201 comprises one or more input devices whereby a user can request motion of the end effector in a desired way. The input devices could, for example, be manually operable mechanical input devices such as control handles or joysticks, or contactless input devices such as optical gesture sensors. The software stored in memory 204 is configured to respond to those inputs and the processor is configured to execute the software to cause the joints of the arm and instrument to move accordingly. In summary, a surgeon at the command interface 201 can control the instrument 103 to move in such a way as to perform a desired surgical procedure. The control unit 202 and/or the command interface 201 may be remote from the arm 102. As explained in more detail below, a load cell unit may be located between the arm and the instrument. Measurements determined by the load cell unit may also be input back into the control unit 202 so as to give the surgeon accurate feedback.

The control unit 202 is configured to translate an input at the command interface 201 to an output at the actuators 404. During an operation, following an input by a surgeon at the command interface, the control unit instructs the actuators to rotate. Rotation of the actuators results in displacement of the drive interface elements. In the example seen in FIG. 4C, each drive interface element comprises a lead screw 406, a ball screw 407 and a ball screw adapter 408. As seen in FIG. 4C, each actuator is coupled to a lead screw 406 such that rotation of the actuator causes rotation of the lead screw about the lead screw's longitudinal axis. The longitudinal axis of the drive interface element 403a is colinear with the longitudinal axis of its respective lead screw. The shaft of the lead screw comprises a threaded portion and a non-threaded portion. A ball screw 407 is threaded onto the threaded portion of the lead screw. The ball screw 407 is restrained, for example by running in a slot defined by a wall of the drive unit, so that it cannot rotate when the lead screw is rotated by the actuator. As a result, rotation of the lead screw by the actuator results in the ball screw translating along the lead screw's longitudinal axis. The ball screw 407 is fixedly secured to a respective ball screw adapter 408. Translation of the ball screw thus results in translation of the ball screw adapter along the longitudinal axis of the drive interface element 403.

FIGS. 4B and 4C also illustrate a key 410 which at one end sits within a slot in the ball screw adapter 408. As seen in FIG. 4B, the slot extends along the outer surface of the ball screw adapter in a direction parallel to the longitudinal direction of the lead screw along which the ball screw adapter translates. The opposite end of the key 410 is secured to the outer wall of the drive unit housing 402. The key cannot move relative to the housing 402. There is a loose-fit connection between the key and ball screw adapter slot such that the key can slide within the slot in the direction parallel to the longitudinal axis of the lead screw. In other words, when the ball screw adapter is actuated by the actuator, lead screw and ball screw to move along the longitudinal axis of the drive interface element 403, the ball screw adapter moves relative to the key. Since the key and the ball screw adapter can move relative to one another in only one direction and cannot rotate relative to one another, the key prevents the ball screw adapter from rotating about its longitudinal axis.

The presence of the key also ensures that the distance between the ball screw adapter and the drive unit housing 402 is fixed. In other words, the key prevents the ball screw adapter from moving in any direction that is not along the longitudinal axis of the drive interface element. Overall, the key acts to prevent unwanted motion of the ball screw adapter.

The key also has the advantage of reducing frictional forces acting during displacement of the ball screw adapter along the longitudinal axis of the drive interface element. With reference to FIG. 4C, the length of the key 410 is greater than the thickness of the wall of the housing 402. The ball screw adapter is thus held by the rotational key in a position in which the outer surface of the ball screw adapter does not make contact with the upper wall of the recess of the housing in which the drive interface element is located. With respect to the upper drive interface element seen in FIGS. 4B and 4C, when the ball screw adapter is translated along the longitudinal axis of the drive interface element, no frictional force acts between the ball screw adapter and the upper wall of the housing recess. Instead, contact is made between the key and the slot in the ball screw adapter as the key slides within the ball screw adapter. The loose-fit connection between the key and ball screw adapter means that friction during this interaction is low.

The drive unit may include four keys, i.e. one key per drive interface element, wherein each key engages the ball screw adapter of its respective drive interface element in the manner described.

In the examples described, the key 410 acts to hold the ball screw adapter away from the outer wall of the recess in the drive unit housing i.e. with respect to the upper drive interface element, to push the ball screw adapter downwards away from the upper portion of the housing. However, in other examples, the key may have another shape, for example an upside down “T” shape, so as to hold the ball screw adapter is a position such that it does not make contact with any of the walls of the recess.

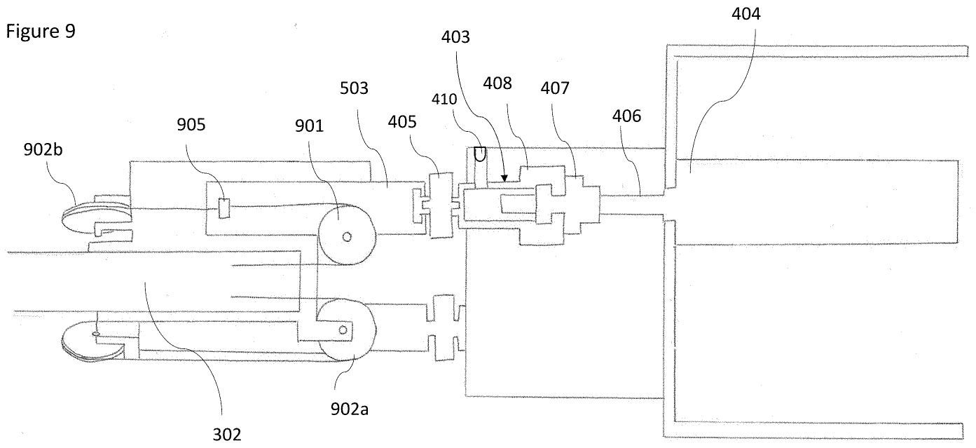

In the embodiment seen in FIG. 4C, each drive interface element can be driven along its respective longitudinal axis by its respective actuator in a direction towards the distal end of the terminal link only. In an alternative example, which will be described in detail below and which is seen in FIG. 9, the drive interface elements can be driven along their respective longitudinal axes both towards and away from the distal end of the terminal link.

As seen in FIG. 4C, the drive unit 401 also comprises a load cell unit 405. The load cell unit is configured to sense the load applied to one or more of the drive interface elements by the actuators. In the example seen in FIG. 4C, the load cell unit senses the load applied to all of the drive interface elements in the drive unit.

The load cell unit comprises four pads, each pad being located at the distal end of a respective drive interface element. Each pad of the load cell unit is configured to sense the load applied to its respective drive interface element by its respective actuator. Displacement of the ball screw adapter 408 results in a force being applied by the ball screw adapter to the pad of the load cell unit 405 and a resulting displacement of the load cell pad. Each ball screw adapter 408 in the example seen in FIG. 4C has an approximately ring-shaped cross section. As such, the ball screw adapter is arranged to make contact with the pad of the load cell unit around the perimeter of the pad. This arrangement means that force from the actuator is applied evenly to the load cell pad i.e. the force is evenly distributed across the cross-sectional area of the load cell pad. This means that the load cell can be flat.

In the example seen in FIG. 4C, a cap 409 is arranged around each pad of the load cell unit. The cap 409 is loosely connected to the ball screw adapter 408 such that some axial play is permitted between the cap 409 and the ball screw adapter 408 of the drive interface element. Accordingly, force imparted onto the ball screw adapter by its respective actuator is transferred first to the pad of the load cell unit 405 and then indirectly to the cap 409. Force is not transferred directly from the ball screw adapter to the cap. The load cell unit therefore accurately senses the load applied to the drive interface element 403 by its respective actuator.

A skilled person would know how to implement a suitable load cell unit for sensing the loads applied to the drive interface elements. For example, the load cell unit may be comprised of one or more strain gauges which output a change in voltage when the one or more strain gauges experience a change in strain due to compression or extension. The change in voltage is translated into the load applied. Alternatively, the load cell may use piezo electric materials to determine the load applied.

As will be explained in more detail below, the force applied to the drive interface elements (which is measured by the load cell unit) corresponds to the tension in the driving elements. In response to receiving feedback from the load cell unit 405, the control unit 202 may be configured to drive the drive interface elements in accordance with the determined tension in the driving elements. For example, parameters relating to the instructed rotation of the actuators may be updated according to the latest driving element tension measurement determined by the load cell unit.

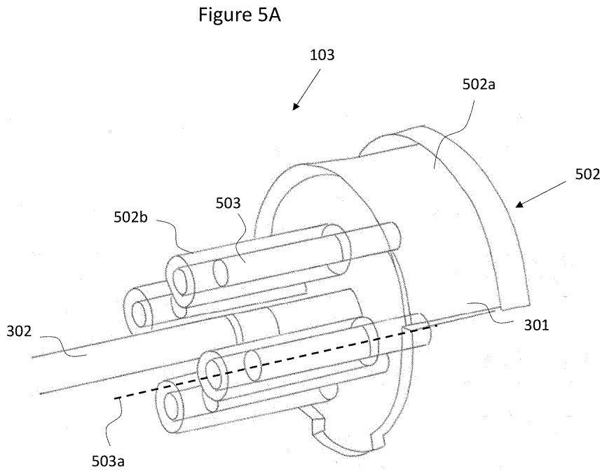

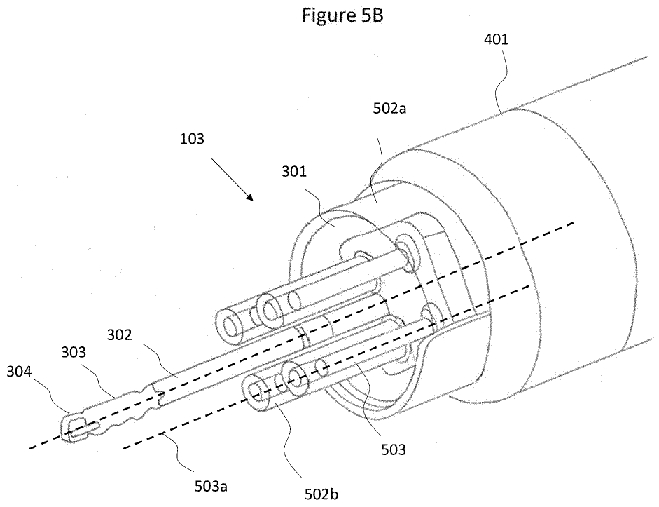

As shown in FIG. 3, the instrument 103 includes instrument interface 301, instrument shaft 302, articulation 303 and end effector 304. FIG. 5A shows a portion of the instrument shaft 302 and the instrument interface 301. The instrument interface comprises a plurality of instrument interface elements 503. In the example seen in FIG. 5A, the instrument interface includes four instrument interface elements 503. In other examples the interface may include more or fewer instrument interface elements. In this example, each instrument interface element is in the form of a rod, e.g. a cylindrical rod. However according to other examples, instrument interface elements may take any other suitable shape. In the example seen in FIG. 5A each instrument interface element has the same shape and dimensions, but in other examples, the instrument interface elements may not be of uniform size and/or shape.

The instrument interface 301 comprises a housing 502. In the example seen in FIG. 5A, the housing comprises a cylindrical cup shaped portion 502a. The housing also comprises a plurality of cylindrical sheaths 502b which protrude from the cup shaped portion. In other examples the housing may take the form of another shape.

The plurality of instrument interface elements are positioned within the housing 502. The instrument interface elements are displaceable. In most displacement positions, the instrument interface elements are positioned partially within the sheaths 502b and partially within the cup shaped portion 502a of the housing. In some displacement positions, the instrument interface elements may be positioned wholly within the sheaths. The instrument interface has at least the same number of sheaths as instrument interface elements. In all displacement positions, each instrument interface element 503 is positioned at least partially within one sheath. Each instrument interface element 503 is configured to move within its respective sheath such that the proportion of the instrument interface element 503 positioned within its respective sheath 502b can be varied. Each instrument interface element 503 has a longitudinal axis 503a. The longitudinal axis of each instrument interface element 503 is aligned with a longitudinal axis of its respective sheath 502b. Each instrument interface element is displaceable along its longitudinal axis. In other examples, the instrument interface elements may be positioned differently within the housing of the instrument interface.

The instrument shaft 302 is affixed to the housing 502 of the instrument interface. The instrument shaft engages a central sheath of the housing. In the example seen in FIGS. 5A and 5B, in which the housing comprises a cylindrical cup shaped portion 502a, the longitudinal axis of the shaft intersects the centre of the cross section of the cup shaped portion. When the instrument interface 301 mates with the drive unit 401 as seen in FIG. 5B, the longitudinal axis of the shaft is colinear with the longitudinal axis of the terminal link of the robot arm. The longitudinal axis of the shaft is parallel to the longitudinal axis of each sheath 502b of the housing and to the longitudinal axis of each of the instrument interface elements 503 located within the sheaths. The direction in which sheaths protrude from the cup shaped portion is parallel to the longitudinal axis of the instrument shaft. In the example seen in FIGS. 5A and 5B, the instrument interface elements are positioned circumferentially around the shaft. In a plane corresponding to a cross section of the cup-shaped portion of the housing 502, each instrument interface element is equidistant from the instrument shaft.

The instrument interface 301 is configured to interface with the drive unit 401. When the instrument 103 is attached to the arm 102, the instrument interface mates with the drive unit. FIG. 6A shows a side view of the instrument interface and drive unit when the two are connected together. The figure shows that the housing 402 of the drive unit mates with the housing 502 of the instrument interface. In this example, the drive unit housing 402 comprises a lip 601 which is engaged by the cup-shaped portion of the instrument interface housing 502 so that the two housings are secured to one another. As previously described, the drive unit comprises four drive interface elements 403 and the instrument interface comprises four instrument interface elements 503. Only two drive interface elements 403 and two instrument interface elements 503 are visible in FIG. 6A. Each drive interface element is positioned adjacent to a respective instrument interface element but is not secured to its respective instrument interface element. Each drive interface element communicates with its respective instrument interface element. FIG. 6A illustrates that the longitudinal axis 403a of each of the drive interface elements is aligned with the longitudinal axis 503a of its respective instrument interface element. In this example, the axes 403a and 503a are colinear. The load cell unit 405 and caps 409 are positioned between the drive interface elements 403 and the instrument interface elements 503. In the example seen in FIG. 6A, each cap 409 is positioned so as to be flush with a face of a pad of the load cell unit on one side and flush with an instrument interface on an opposite side. As previously explained, a displacement of the ball screw adapter 408 of the drive interface 403 results in a displacement of a pad of the load cell 405 and of the corresponding cap 409. In this example, each pad of the load cell unit 405 is secured to the drive interface elements 403 by a cap 409 but is not secured to the instrument interface elements. Each cap 409 is not secured to any of the instrument interface elements. The drive interface elements are not otherwise secured to the instrument interface elements. In other examples, the load cell unit is located between but not secured to either the drive interface elements or the instrument interface elements. This arrangement is advantageous as there will be no degradation of any securement mechanism (for example glue or a screw) between the load cell unit and the drive interface elements over time. Thus in the arrangement in which the load cell unit is not secured to either the drive interface elements or the instrument interface elements the coupling between the load cell and the interface elements will maintain its stiffness over time. As previously explained, each drive interface element can be displaced by its respective actuator. Displacement of the drive interface element is along its longitudinal axis in a direction towards the distal end of the terminal link, i.e. towards the end effector. As previously mentioned, neither the drive interface element nor the load cell unit are secured to the respective instrument interface element. However, when the drive interface element is displaced towards its distal end, because the components of the drive unit, the load cell unit and instrument interface are positioned flush to one another and aligned along the longitudinal axis 403a (and 503a), when the drive interface element is pushed, it pushes the load cell unit which pushes the instrument interface element (optionally via a cap 409). A displacement of the drive interface element in said direction thus results in an equal displacement of the pad of the load cell unit, which results in an equal displacement in the same direction of the corresponding instrument interface element. In other words, a displacement of a drive interface element along its longitudinal axis towards its distal end results in a displacement of its respective instrument interface element along the longitudinal axis of the instrument interface element towards the distal end of the instrument interface element. It is noted again that the longitudinal axis of the instrument interface element is colinear with the longitudinal axis of the respective drive interface element. It is also noted that a direction towards the drive interface element's distal end is the same as the direction towards the instrument interface element's distal end, both directions being towards the end effector.

This transfer of displacement along the same axis results in efficient force transfer between the drive unit and the instrument. Compared with previous arrangements, in which the drive interface elements are not driven along the same line as their respective instrument interface elements, the arrangement described above results in reduced energy losses. Furthermore, because the pads of the load cell unit are also positioned along the same line, the load cell is able to more accurately determine the force that has been applied to each of the instrument interface elements.

Finally, since the longitudinal axis of each drive interface/instrument interface element pair is positioned so close to the instrument shaft (as seen in FIGS. 5A, 5B and 6), the overall drive unit instrument interface is more compact.

In some examples described herein, since the drive interface elements are not attached to their respective instrument interface elements, each instrument interface element can only be pushed (but not pulled) by its respective drive interface element. The instrument interface elements can thus only be displaced in one direction by driving the corresponding drive interface elements. In particular, each instrument interface element can only be displaced towards its distal end by driving the corresponding drive interface elements. This is the direction towards the end effector. Since none of the instrument interface elements are secured to any of the drive interface elements or the load cell unit, the drive interface elements are not capable of “pulling” the instrument interface elements. Therefore, even if the drive interface elements could be displaced both towards and away from the end effector, displacement of the drive interface element in a direction away from the end effector could not be transferred to a displacement of the corresponding instrument interface element.

As will be explained in detail below, it is possible to effectively control the position of the end effector in these “push-only” examples using four (or more) driving elements, each driving element being coupled to a respective instrument interface element. In these “push-only” examples, total tension is shared across the four driving elements such that the tension in one driving element is not independent of tension in all the other driving elements. An instrument interface element may therefore be displaced in a direction towards its proximal end due to changes of tension in driving elements which are not coupled to the respective instrument interface element (provided the pushing force previously applied by the drive interface element to the instrument interface element is reduced or removed). As described in more detail below, tension in the respective driving element may therefore be altered by pushing on other ones of the drive interface elements.

Furthermore, there are numerous advantages to the arrangement seen in FIG. 6A. The fact that the drive interface elements are not secured to the instrument interface elements means that attaching the instrument interface of the instrument onto the drive unit of the robot arm is simple and quick. Exact alignment of each instrument interface element with its corresponding drive interface element is not initially essential during attachment as the cup shaped portion 502a of the instrument interface housing and lip on the drive unit housing allows for a degree of uncertainty in the respective positions of the drive unit and instrument interface during the initial attachment process. Furthermore, the lack of attachment between the respective interface elements facilitates removal of the instrument from the robot arm. Removal of the instrument from the robot arm requires only that the tension in the driving elements is reduced to zero. This is advantageous in situations in which power to arm is lost and the instrument is to be detached from the arm. Upon a loss in power, tension in the driving element is lost meaning that the instrument can be removed from the robot arm.

In further examples, the drive unit may not include a load cell unit. In such an example, each drive interface element is positioned so as to be flush with its respective instrument interface element. Displacement of the drive interface element in a direction towards the distal end of the drive interface element is therefore translated directly into displacement of the instrument interface element.

A drape cup 602 is shown in FIG. 6B. The drape cup shown includes a rigid outer portion and a central flexible or folding portion. The drape cup may be positioned between and engage both the drive unit 401 and the instrument interface 301 as seen in FIG. 6C. The central portion of the drape cup may have multiple flexible portions which can move relative to one another. Each of the flexible portions may be located between a drive interface element and an instrument interface element so as not to affect the transfer of displacement of the drive interface elements to the instrument interface elements. The drape cup may be attached to a surgical drape 603 such that the drape separates the robot arm 102 from the instrument 103. The drape may cover the arm so as to maintain a sterile barrier between the arm and the instrument.

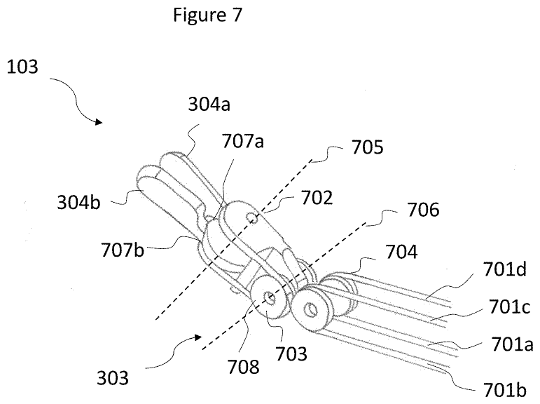

FIG. 7 shows the distal end of the instrument shaft 302 which includes articulation 303 and end effector 304. In this example, the end effector 304 comprises two smooth jaws 304a and 304b. In other examples, the end effector may be serrated jaws, a gripper, a pair of shears, needle graspers, biopsy needles or needles for injecting drugs, or any other suitable end effector for a surgical instrument. The articulation 303 comprises several joints which permit the end effector to move relative to the shaft of the instrument. The joints in the articulation are actuated by driving elements 701, such as cables. At the distal end of the instrument shaft 302, the driving elements 701 engage the joints in the articulation. At the proximal end of the shaft, the driving elements are secured to interface elements 503 of the instrument interface. Each instrument interface element is secured to a respective driving element.

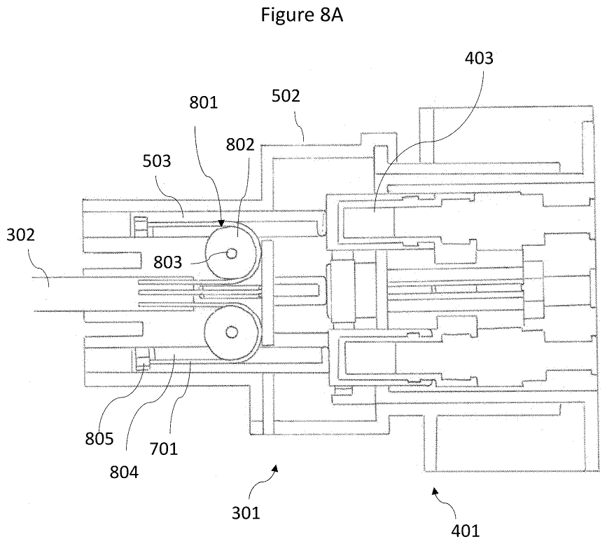

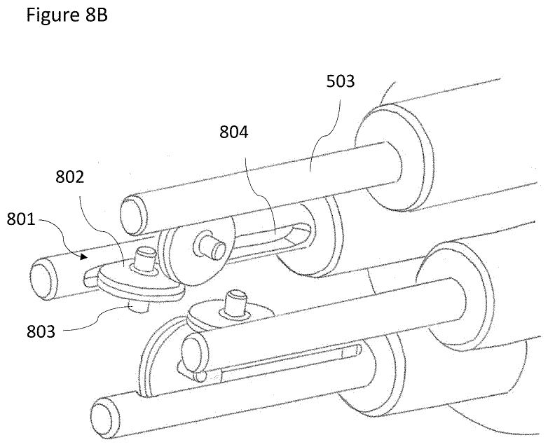

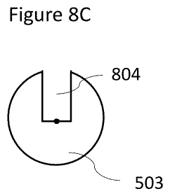

FIG. 8A shows a side view of the instrument interface 301 and part of drive unit 401 when the instrument interface and drive unit are mated. As previously described, in this example the drive unit comprises four drive interface elements 403 and the instrument interface comprises four instrument interface elements 503. Only two of the four drive interface elements 403 and two of the four instrument interface elements 503 are visible in FIG. 8A. The figure illustrates an example of how each instrument interface element may be secured to its respective driving element.

In the example seen in FIGS. 8A to 8C, each instrument interface element is a cylindrical rod. Each instrument interface element 503 comprises a groove 804 which extends at least partially along the length of the rod. The groove has an elongate shape which is parallel to the rod. The groove has a depth which is substantially equal to the radius of the rod. In other words, the groove also extends generally along the radius of the instrument interface element, i.e. from the edge to the centre of the cross section of the instrument interface element, as seen in FIGS. 8B and 8C. Each instrument interface element is associated with a respective pulley 801 which is also positioned within housing 502, as seen in FIG. 8A. In this example, each pulley 801 comprises a main body 802 which has a circular and largely planar shape. Each pulley also comprises an axle 803 about which the main body 802 of the pulley can rotate. In other examples, the pulley may be a capstan. Returning to the example seen in FIGS. 8A and 8B, the main body of each pulley is positioned at least partially within the groove 804 of the respective instrument interface element. The pulley does not contact the instrument interface element or the groove. The plane of the main body of each pulley is parallel to the groove formed in its respective instrument interface element. The main body of the pulley may rotate within the groove, i.e. the main body of the pulley can rotate relative to the instrument interface element. Each pulley (both the main body 802 and the axle 803) is fixed with respect to the instrument shaft 302 but the main body 802 is rotatable relative to the instrument shaft. Each pulley is displaceable relative to its respective instrument interface element in a direction parallel to the instrument shaft and parallel to the longitudinal axis of the respective instrument interface element. Specifically, each pulley can be displaced within the groove in which it is positioned in a direction parallel to the longitudinal axis of the respective instrument interface element.

The main body 802 of each pulley is positioned (or “seated”) in the groove of its respective instrument interface element such that the plane of the main body is parallel to the longitudinal axis of the respective instrument interface element. The edge of the pulley meets the longitudinal axis of the instrument interface element, i.e. the edge of the pulley is positioned in the centre of the cross section of the instrument interface element, as seen in FIG. 8A. To put it another way, a tangent of the edge of the main body of the pulley is colinear with the longitudinal axis of the instrument. In other words, a position at one end of a diameter of the main body of the pulley is aligned with the longitudinal axis of the respective instrument interface element.

The other end of the diameter of the main body of the pulley is located within a projected profile of the instrument shaft i.e. within a volume that would have been occupied by the shaft had the shaft continued to extend towards the drive unit. In other words, since the instrument shaft has a constant circular cross section, the other end of the diameter of the main body of the pulley is located within a “virtual” cylinder having the same cross section and longitudinal axis as the shaft, but in a location closer the drive unit.

The axle of each pulley is not positioned within the groove of the respective instrument interface element. The axle of each pulley is positioned outside the volume formed by its respective instrument interface element. The axle of each pulley is perpendicular to the longitudinal axis of its respective instrument interface element.

FIG. 8B shows all four instrument interface elements but omits the instrument shaft. As previously explained, in this example, the instrument interface elements are positioned circumferentially around the instrument shaft (not shown). The groove in each instrument interface element is positioned on the side of the instrument interface element which is closest to the instrument shaft. In other words, the groove faces the instrument shaft. Thus, each pulley is positioned between its respective instrument interface element and the instrument shaft. The plane of the main body of each pulley is parallel to the longitudinal axis of the instrument shaft. As seen in FIG. 8A, the main body of each pulley extends between the longitudinal axis of its respective instrument interface element and a position that is within the cross-section of the instrument shaft when looking down the longitudinal axis of the instrument shaft. As explained above, one end of the diameter of the main body of the pulley is aligned with the longitudinal axis of its respective instrument interface element. The other end of the diameter of the main body of the pulley (i.e. an opposite edge of the pulley) is located close to the longitudinal axis of the instrument shaft, for example the main body of the pulley may intersect the shaft's longitudinal axis or be otherwise positioned within a projection of the volume of the instrument shaft along the longitudinal axis of the instrument shaft. In the example seen in FIG. 8A, the other end of the diameter is closer than the edge of the instrument shaft to the longitudinal axis of the instrument shaft, but does not intersect the longitudinal axis of the instrument shaft.

The instrument interface elements are secured to driving elements 701 used in the articulation at the distal end of the instrument shaft. The instrument comprises four driving elements 701, but only two driving elements of the four are seen in FIG. 8A. Each instrument interface element is secured to a respective driving element. In the example seen in FIG. 8A, each instrument interface element is attached to a driving element 701 towards the distal end of the instrument interface element. Each instrument interface element is attached to a driving element 701 at a position that is more distal than the axle of the corresponding pulley 801. This means that tension in the driving element 701 will act to pull the instrument interface element in a direction towards the proximal end of the instrument interface element. FIG. 8A shows that each driving element is secured to its respective instrument interface element using a clip 805 but other attachment means may be used. In this example, each instrument interface element is secured to a single length of a driving element. At one end of its the length, the driving element is secured to its respective instrument interface element using a clip 805. The clip 805 is located within the groove 804 such that the driving element is also positioned in the groove. The groove may include features designed to increase friction between the groove and the driving element. For example, the groove may be sized so as to maximise friction between the groove and the driving element. The groove may be made from a high friction material or have a coating designed to increase grip on the driving element. In one example, the groove may comprise one higher friction material or coating in areas which engage the driving element and one lower friction material or coating in areas which engage the main body of the pulley meaning that the pulley is able to rotate freely within the groove. The driving element extends along the groove away from the clip, in the direction towards the proximal end of the instrument interface element and engages the respective pulley also present in the groove. The driving element engages the main body of the pulley at the point on the main body's edge which is aligned with the longitudinal axis of the instrument interface element. In one example, a portion of the driving element is secured to the main body of the pulley, for example using a crimped bead or other securement mechanism such that displacement of the driving element causes a rotation of the main body of the pulley. In other examples, the driving element is not secured to the pulley and so that the driving element can move relative to the main body of the pulley and does not rotate the pulley as it is displaced. In this example, the main body of the pulley may have a coating configured to reduce frictional forces acting between the pulley and the driving element. The driving element extends around the respective pulley 801 such that the pulley engages the driving element along half of its circumference. In other words, the driving element wraps around half the circumference of the pulley and then disengages from the pulley. The direction of the driving element when it disengages from the pulley is therefore in the direction towards the distal end of the instrument interface element. The driving element disengages from the main body of the pulley at the point on the main body which is closest to the longitudinal axis of the instrument shaft and enters the instrument shaft. The driving element then extends within the instrument shaft along the shaft towards the shaft's distal end (not shown in FIG. 8A). At the distal end of the instrument shaft, the driving element engages with the articulation 303, as shown in FIG. 7.

As previously explained, an input at the command interface 201 results in a displacement of one or more drive interface elements 403 of the drive interface on the robot arm's terminal link. When an instrument is attached to the terminal link, a displacement of a drive interface element along its longitudinal axis towards the distal end of the drive interface element results in displacement of its respective instrument interface element 503 in the same direction and along the same axis.

As explained above, each driving element is secured at one of its ends to the distal end of a respective instrument interface element. A displacement of the instrument interface element in the direction towards its distal end therefore results in an equal displacement of the end of the driving element in the same direction. Since the driving element is secured to the main body of the pulley and the main body of the pulley can be rotated within the groove of its respective instrument interface element, displacement of the driving element along the longitudinal axis of the instrument interface element (i.e. in a direction perpendicular to the axle of the pulley) causes the main body of the pulley to rotate about the pulley's axle. Because the driving element wraps partially around the pulley, the portion of the driving element which is positioned within the instrument shaft is displaced in the opposite direction, towards the proximal end of the instrument interface element, i.e. away from the end effector. The displacement of the drive interface element in a direction towards the end effector therefore results in a displacement of the corresponding driving element in the opposite direction, away from the end effector.

It is known that frictional losses occur when driving elements are engaged by pulleys. In examples where the driving element is not secured to its respective pulley and can move relative to the main body of the pulley, frictional losses are increased as the radius of the pulley decreases. Furthermore, the radius of the pulley which is engaged by the driving element has a significant effect on the wear of the driving element due to the fact that the driving element is required to engage with a larger proportion of the circumference of the pulley and therefore change direction more sharply in order to wrap around the pulley. A pulley with a smaller radius can therefore lead to a concentration of stress and faster wear on the driving element. It thus desirable to maximise the radius of any pulley engaged by a driving element so as to reduce sharp changes of direction in the path of the driving element. Furthermore, it is generally desirable to minimise the number of pulleys that a driving element is required to engage.

The arrangement seen in FIGS. 8A and 8B allows for each driving element to engage only a single pulley in the instrument interface. In other words, each driving element engages only one pulley between being engaged by an instrument interface element and entering the instrument shaft. This is an improvement compared with earlier instrument interface arrangements. Furthermore, the fact that each pulley 801 is positioned within a groove in an instrument interface element means that the radius of the pulley can be maximised while minimising the distance between the instrument interface elements and the shaft, thereby minimising the overall size of the instrument interface.

In addition, because each driving element attaches to its respective instrument interface element at a point along the instrument interface element's longitudinal axis and is driven along that axis, which is also the axis along which both the drive interface element and instrument interface element are displaced, a further reduction in energy loss in the transfer of force from the drive interface element to the driving element is enabled. The transfer of force from the drive interface elements to the end effector is thus made more efficient while maintaining an overall compact instrument interface. Furthermore, because the driving element, instrument interface element and drive interface element are displaced along the same line of action and the load cell unit is also positioned on that line of action, the load cell is able to accurately measure the tension in the driving element.

In an alternative example, each pulley is not located within a groove of the instrument interface element. In this arrangement, the driving element would not be driven along the same axis as the instrument interface element. This arrangement would enable force to be transferred from the drive interface element to the driving element but the force transfer would be less efficient.

As seen in FIG. 7, at the distal end of the instrument shaft 302, the driving elements 701 engage joints in articulation 303 and are used to actuate motion of the end effector 304. In this example, the end effector comprises two end effector elements 304a and 304b. Each end effector element comprises a jaw and a circular portion. The articulation is formed of a first joint 702, a second joint 703 and a further set of pulleys 704.

The first joint 702 has a first axis 705, the yaw axis. The first axis 705 is perpendicular to the longitudinal axis of the instrument. In the example seen in FIG. 7, the first joint comprises two pulleys 707 which are disposed about the first axis 705. The circular portion of each end effector element is also disposed about the first axis 705. The circular portion of each end effector element can be rotated about the first axis such that the end effector is rotated about the first axis. Each pulley of the first joint is rotationally fixed to the circular portion of an end effector element. Therefore, a rotation of a pulley 707 of the first joint about the first axis causes equal rotation of its corresponding end effector element about the first axis i.e. yaw of the end effector element. If this rotation is in a direction away from the other end effector element, said rotation results in an opening of the end effector jaws. By rotating both end effector elements about the first axis in the same direction, the end effector (both end effector elements) can undergo yaw rotation. Similarly, by rotating the two end effector elements about the first axis in opposite directions, the jaws of the end effector can be opened and closed.

The second joint 703 has a second axis 706, the pitch axis. The second axis 706 is perpendicular to the longitudinal axis of the instrument. The second axis 706 is perpendicular to the first axis 705. The end effector can be rotated about the second axis 706 to undergo pitch motion. In the example seen in FIG. 7, the second joint comprises four pulleys 708 which are disposed about the second joint.

The second joint 703 is positioned between the first joint 702 and the set of further pulleys 704. In the example seen in FIG. 7, the set of further pulleys is formed of four further pulleys. The further pulleys are all disposed about a single axis which is parallel to the second axis.

The position of the end effector is controlled using four driving elements. In some examples, each driving element may be unconnected to any other driving element. In other examples, each driving element 701 forms a pair of driving elements with another driving element such that the instrument is controlled using two pairs of driving elements. In some examples, each pair of driving elements is formed of two driving elements which are not connected to one another. However, in the following example, each driving element 701 joins one other driving to form a pair. In other words, each driving element is one end of a pair of driving elements. The proximal end of each driving element engages an instrument interface element in the instrument interface. The distal end of each driving element engages another driving element to form a pair of driving elements. The example seen in FIG. 7 has two pairs of driving elements. The first pair of driving elements 701ab is formed of driving elements 701a and 701b. The second pair of driving elements 701cd is formed of driving elements 701c and 701d.

Each pair of driving elements is secured to a pulley 707 of the first joint. The pair of driving elements may be secured to the pulley by a bead which has been crimped or by any other securement mechanism. The tension in one driving element of the pair is not transferred to the tension in the other driving element of the pair. As previously mentioned, the tension in one driving element pair is transferred to the other pairs of driving elements. In an alternative example in which the driving elements do not form pairs, each driving element may be secured to a pulley of the first joint.

Referring to the first pair of driving elements 701ab, the path of the pair of driving elements starting from the proximal end of the articulation is as follows. Having extended along the instrument shaft, the pair engages a pulley 704 of the set of further pulleys, engages a pulley 708 of the second joint, then following a direction generally towards the distal end of the instrument, wraps half the circumference of a pulley 707 of the first joint so that the direction of the pair of driving elements is reversed. As previously explained, the pair of driving elements is secured to the pulley 707 of the first joint. The pair then follows a path in the direction away from the distal end of the instrument, engages another pulley 708 of the second joint on the other side of the instrument and engages another further pulley 704 before extending along the instrument shaft towards the proximal end of the instrument. The other pair of driving elements 701cd follows an equivalent and complementary path. The paths taken by the two pairs of driving elements are symmetrical about a plane which includes both the first axis 705 and the longitudinal axis of the instrument when the instrument has a straight configuration.

As previously explained, displacement of a drive interface element in a direction towards the end effector results in a displacement of a corresponding driving element within the instrument shaft in the opposite direction, away from the end effector. In this example, each driving element can be driven in only one direction by its corresponding drive interface element, away from the end effector (i.e. the driving element is pulled). However, since the tension in one pair of driving elements is not independent of the tension in all the other pairs of driving elements, the driving element can move in the opposite direction due to changes in tension of the other pairs of driving elements.

A displacement of a driving element in the following explanation always means a displacement in the direction away from the distal end of the instrument (i.e. a displacement which is driven by the respective actuator, drive interface element and instrument interface element). The motion of the end effector can be controlled using the four driving elements 701a, 701b, 701c and 701d (forming pairs of driving elements 701ab and 701cd) as follows.

A displacement of driving element 701a causes a rotation of the pulley 707a to which it is secured and thus the end effector element 304a (to which the pulley is secured) in a direction away from the other end effector element 304b (the clockwise direction in FIG. 7). A displacement of driving element 701a therefore causes the end effector jaws to open.

A displacement of driving element 701b causes a rotation of the pulley 707a and end effector element 304a in a direction towards the other end effector element 304b (the anticlockwise direction in FIG. 7). A displacement of driving element 701b therefore causes the end effector jaws to close.

A displacement of both driving element 701a and 701b at the same time causes a rotation of the end effector about the second axis 706, i.e. a simultaneous displacement of driving elements 701a and 701b causes a pitch motion of the end effector in the clockwise direction seen in FIG. 7. A displacement of just one of driving elements 701a or 701b thus also contributes to a (lesser) pitch motion as the point of securement to the driving element to the pulley 7071 is offset from the axis 706 about which the end effector pitches.

A displacement of driving element 701c causes a rotation of the pulley 707b to which it is secured and thus the end effector element 304b (to which the pulley is secured) in a direction away from the other end effector element 304a (the anticlockwise direction in FIG. 7). A displacement of driving element 701c therefore causes the end effector jaws to open. A simultaneous displacement of driving element 701a and 701c therefore result in a symmetrical and quicker opening of the end effector jaws.

A displacement of driving element 701d causes a rotation of the pulley 707b and end effector element 304b in a direction towards the other end effector element 304b (the clockwise direction in FIG. 7). A displacement of driving element 701d therefore causes the end effector jaws to close. A simultaneous displacement of driving element 701b and 701d therefore result in a symmetrical and quicker closing of the end effector jaws.

A displacement of both driving element 701c and 701d at the same time causes a rotation of the end effector about the second axis, i.e. a simultaneous displacement of driving elements 701c and 701d causes a pitch motion of the end effector in the anticlockwise direction seen in FIG. 7.

Since a displacement of driving element 701a causes a rotation of end effector element 304a in the clockwise direction and a displacement of driving element 701d causes a rotation of end effector element 304b in the clockwise direction, the end effector as a whole can be made to rotate in the clockwise direction about the first axis by a simultaneous displacement of driving elements 701a and 701d. In other words, driving elements 701a and 701d can be displaced at the same time to cause a yaw rotation of the end effector in one direction.

Since a displacement of driving element 701b causes a rotation of end effector element 304a in the anticlockwise direction and a displacement of driving element 701c causes a rotation of end effector element 304b in the anticlockwise direction, the end effector as a whole can be made to rotate in the anticlockwise direction about the first axis by a simultaneous displacement of driving elements 701b and 701c. In other words, driving elements 701b and 701c can be displaced at the same time to cause a yaw rotation of the end effector in an opposite direction.

In this way, four driving elements can be used to control pitch motion, yaw motion and opening and closing of the jaws of the end effector.

In one example operation, in which the surgeon wishes to open the jaws of the end effector, the surgeon will provide an input at the command interface 201 instructing the robot to open the end effector jaws. The control unit 202 will translate the surgeon's input at the command interface 201 to an output at the appropriate actuators 404. In this example, the control unit will instruct two actuators 404 to rotate. The two appropriate actuators will be those which correspond to driving elements 701a and 701c of the instrument.

As previously explained, a rotation of an actuator will cause a displacement of its respective drive interface element 403 along the longitudinal axis of that drive interface element. Rotation of two actuators will therefore cause a displacement of two drive interface elements. Each drive interface element communicates with a respective instrument interface element 503 such that displacement of a drive interface element causes displacement of the corresponding instrument interface element along the same axis. The displacement of two drive interface elements will therefore result in displacement of two respective instrument interface elements in a direction towards the distal end of the instrument along the longitudinal axis of the respective instrument interface element. As previously explained with regard to FIGS. 8A and 8B, a driving element is secured to each instrument interface element such that a displacement of the instrument interface element in the direction towards the instrument's distal end results in a displacement of the respective driving element in the direction away from the instrument's distal end. The displacement of the two instrument interface elements will therefore result in simultaneous displacement of driving elements 701a and 701c. As previously explained, displacing these driving elements at the same time will result in the end effector elements rotating away from one another i.e. the instrument jaws opening.

As previously discussed, tension is shared between the pairs of driving elements such that they compete against each other for tension i.e. tension in one pair of driving elements is transferred to all the other pairs of driving elements through the end effector elements. The driving elements can therefore be simultaneously tensioned by displacing them all at the same time. By pulling on all of the driving elements simultaneously using the same force, the tension in all the driving elements is increased but the position of the end effector remains unchanged. In other words, to increase the tension in all of the driving elements, the surgeon can provide an input at the command interface 201 which will cause all the actuators to rotate thereby displacing all the drive interface elements and all the instrument interface elements. As previously explained, the load cell unit can determine accurate measurements of the tension in the driving elements and these measurements may be input back into the control unit 202 so as to give the surgeon accurate feedback. This feedback can be used by the surgeon to determine whether the driving elements have reached the desired tension or whether they should be re-tensioned. All driving elements can be tensioned at any point during a surgical procedure without affecting the position of the end effector. This arrangement is advantageous as it is not required to pre-tension the driving elements during set up when attaching the instrument to the robot arm. A further advantage is that parameters used in controlling the instrument, for example the torque delivered by the actuators, may be updated according to the feedback i.e. in response to updated values for the tension in the driving elements.

In the embodiment described above, each of the drive interface elements 403 is not attached to a respective instrument interface element but pushes the respective instrument interface element 503 such that a displacement of the drive interface element results in a displacement of the respective instrument interface element. None of the drive interface elements are secured to any of the instrument interface elements. Each instrument interface element can therefore only be pushed by its respective drive interface element and so can only be displaced by its respective drive interface element in one direction, the direction towards the end effector. As previously described, each instrument interface element is secured to a respective driving element such that a displacement of the instrument interface element in a first direction results in a displacement of the respective driving element in the opposite direction. Each driving element can therefore also only be driven by the actuators to be displaced in one direction, away from the end effector.

FIG. 9 shows an alternative embodiment in which each drive interface element 403 is attached to its respective instrument interface element 503 via the load cell unit 405. In this embodiment, the drive interface includes three drive interface elements which are each driven by a single actuator such that the drive interface also includes only three actuators. FIG. 9 shows only one of the three drive interface elements 403 and one of the three actuators 404. In this embodiment, each drive interface element can be displaced in two opposite directions by its respective actuator. Due to the attachment between the drive interface elements and the instrument interface elements, each drive interface element can transfer that displacement to the respective instrument interface element so that the instrument interface element can also be displaced in two opposite directions (both towards and away from the end effector). The operation of the actuator 404, lead screw 406 and ball screw 407 is the same as previously described. The drive unit also includes keys 410 for controlling the motion of the ball screw adapters as previously described. The ball screw adapter is as previously described but additionally can be controlled to move in two opposite directions (push and pull). The ball screw adapter 408 seen in FIG. 9 has a slightly different shape than the ball screw adapter previously described as the ball screw engages the ball screw adapter so that the ball screw adapter can be pushed and pulled by the ball screw. Furthermore, unlike the example previously described, the ball screw adapter 408 is secured to the load cell unit 405. The load cell unit is secured to both the ball screw adapter 408 of the drive interface element and to the respective instrument interface element 302. The load cell unit in FIG. 9 determines the force applied to the instrument interface element and thus the tension in the corresponding driving element in two opposite directions (towards and away from the end effector). The load cell unit may be configured to sense tension and compression. Unlike the previous embodiments, the load cell unit in this example is secured to both the drive interface element and the instrument interface element. In the example shown in FIG. 9, the load cell unit may be secured to the drive interface element and the instrument interface element by means of screw fits. Due to the connection between the load cell unit with the drive interface element and instrument interface element, force applied to the ball screw adapter is applied directly to the load cell unit 405 which is transferred to the instrument interface element 302. When the actuator initiates a displacement of the ball screw adapter 408 in the direction away from the end effector (i.e. when the drive interface element is pulled), the “T” shaped engagement element between the load cell unit 405 and the instrument interface element 302 seen in FIG. 9 means that the displacement is transferred to an equivalent displacement in the same direction of the instrument interface element.

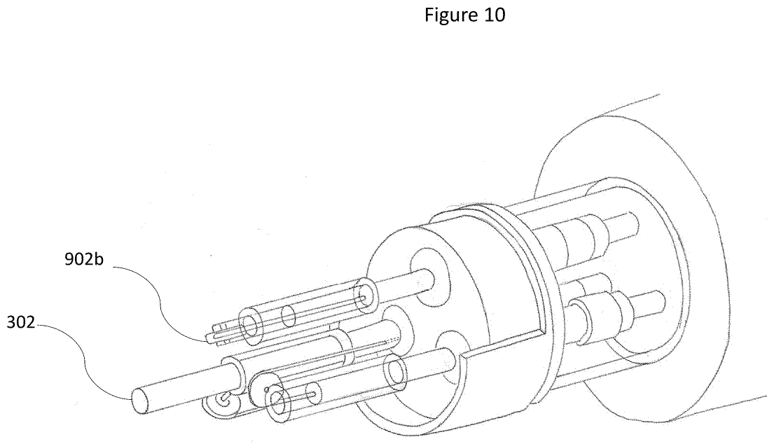

In this embodiment, the drive interface includes only three drive interface elements, each drive interface element being attached to one instrument interface element in the instrument interface. The instrument interface therefore includes only three instrument interface elements. FIG. 10 shows that the instrument interface elements are disposed circumferentially around the instrument shaft. The angular displacement between neighbouring instrument interface elements is 120 degrees. In contrast to the embodiment seen in FIGS. 4 to 8, in this alternative embodiment, each driving element is a loop of cable, rather than a single length of cable. The instrument therefore includes three cable loops, each loop engaging an instrument interface element and a pulley of a joint in the articulation.

FIGS. 9 and 10 illustrate the engagement between the cable loops and their respective instrument interface elements. In a similar way to that previously described, a point on the cable loop is secured to its respective instrument interface element by a clip 905 or other securement mechanism.

In contrast to the previous embodiment, each instrument interface element is associated with three respective pulleys 901, 902a, 902b such that the instrument interface includes a total of nine pulleys. The first of the three pulleys 901 is equivalent to one of the pulleys seen in FIGS. 8A and 8B. As previously described and seen in FIG. 9, each pulley 901 is located partially within a groove of its respective instrument interface element and engages a portion of the cable loop which is driven by the instrument interface element in the manner previously described. The pulleys 901 enable a displacement of the respective instrument interface element in a direction towards the end effector to be translated into a displacement of a portion of the cable loop in a direction away from the end effector i.e. to enable a cable loop “pull” to occur.

Each instrument interface element is associated with two further pulleys, a proximal pulley 902a and a distal pulley 902b. The distal pulley 902b is located at the distal end of its instrument interface element. FIG. 10 shows only the distal pulleys 902b. The axel of the pulley 902b intersects and is perpendicular to the longitudinal axis of the instrument shaft. The plane of the main body of the pulley 902b is parallel to a tangent to the instrument shaft.

The proximal pulley 902a is positioned adjacent to its respective instrument interface element towards the proximal end of the instrument interface element. Specifically, the pulley 902a is located between the instrument interface element and a neighbouring instrument interface element. The plane of the main body of the proximal pulley 902a is colinear with a radius of the instrument shaft. In other words, the three proximal pulleys are positioned radially around the instrument shaft 302. Thus, for a particular instrument interface element, its proximal pulley 902a is perpendicular to its distal pulley 902b. The pulleys 902a and 902b enable a displacement of the respective instrument interface element in a direction away from the end effector to be translated into a displacement of a portion of the cable loop in a direction towards the end effector i.e. to enable a cable loop “push” to occur.

The proximal pulley 902a and distal pulley 902b engage the cable loop. As seen in FIG. 9, the portion of the cable loop attached to the instrument interface element by clip 905 extends towards the distal end of the instrument interface element towards the distal pulley 902b and wraps around half of the circumference of the distal pulley. The path of the cable loop then extends away from the distal pulley in a direction towards the proximal end of the instrument interface element and engages the proximal pulley 902a. The cable loop wraps around half of the circumference of the proximal pulley and enters the instrument shaft. As seen in FIG. 9, for each instrument interface element, two portions of the cable loop (from pulleys 901 and 902a) extend along the instrument shaft towards its distal end. The articulation at the distal end of the instrument shaft is therefore controlled using three loops of cable. The skilled person would know how to use these three cable loops to actuate an end effector at the distal end of an instrument.

In both embodiments described above, the displacement of each drive interface element, its respective instrument interface and driving element is along the same axis, resulting in efficient force transfer. Since the load cell unit is also positioned on this axis, determination of the tension in said driving element is highly accurate. As explained above, the load cell unit in FIG. 9 is configured to determine changes of tension corresponding to displacement of driving elements in both directions i.e. both towards and away from the end effector. This embodiment is also advantageous as fewer actuators, interface elements and driving elements are required to control motion of the end effector than in the embodiment seen in FIGS. 4 to 8.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

What is claimed is:

1. A drive unit for a surgical robot arm, the robot arm being configured to engage a robotic surgical instrument, the drive unit comprising:
a plurality of drive interface elements, each drive interface element having a longitudinal axis; and
a plurality of actuators configured to drive the plurality of drive interface elements, each actuator of the plurality of actuators being configured to drive one of the plurality of drive interface elements so as to cause that drive interface element to be displaced along its longitudinal axis in a first direction,
wherein the drive unit is configured such that, when the surgical robot arm engages the robotic surgical instrument, the longitudinal axis of each drive interface element is aligned with a longitudinal axis of a respective instrument interface element in the instrument and each drive interface element is configured such that the displacement of said drive interface element along its longitudinal axis in the first direction causes a displacement of the respective instrument interface element along its longitudinal axis in the first direction.

2. The drive unit of claim 1, wherein the drive unit is configured such that, when the surgical robot arm engages the robotic surgical instrument, the longitudinal axis of each drive interface element is collinear with the longitudinal axis of the respective instrument interface element in the instrument.

3. The drive unit of claim 1, wherein each drive interface element has a proximal end and a distal end and the first direction extends from the proximal end to the distal end.

4. The drive unit of claim 1, wherein the drive unit is configured such that, when the surgical robot arm engages the robotic surgical instrument, the plurality of drive interface elements are not secured to the respective instrument interface elements in the instrument.

5. The drive unit of claim 1, wherein the drive unit comprises a load cell unit configured to sense a load applied to one or more of the plurality of drive interface elements by the plurality of actuators, and wherein the drive unit is configured such that, when the surgical robot arm engages the robotic surgical instrument, the load cell unit is positioned between the plurality of drive interface elements and the respective instrument interface elements in the instrument.

6. (canceled)

7. The drive unit of claim 56, wherein the load cell unit comprises a plurality of pads, and the drive unit is configured such that, when the surgical robot arm engages the robotic surgical instrument, each of the pads are positioned such that the load cell unit transfers the displacement of each drive interface element along its longitudinal axis in the first direction to a displacement of the respective instrument interface element along its longitudinal axis in the first direction.

8. The drive unit of claim 1, wherein each actuator of the plurality of actuators is configured to drive a drive interface element so as to cause that drive interface element to be displaced along its longitudinal axis in the first direction only.

9. The drive unit of claim 1, wherein each actuator of the plurality of actuators is configured to drive a drive interface element so as to cause that drive interface element to be displaced along its longitudinal axis in the first direction and in a second direction, wherein the second direction is opposite to the first direction.

10. The drive unit of claim 9, the drive unit being configured such that, when the surgical robot arm engages the robotic surgical instrument, the displacement of a drive interface element along its longitudinal axis in the second direction does not cause a displacement of the respective instrument interface element in the instrument.

11. The drive unit of claim 9, the drive unit being configured such that, when the surgical robot arm engages the robotic surgical instrument, the displacement of a drive interface element along its longitudinal axis in the second direction causes a displacement of the respective instrument interface element along its longitudinal axis in the second direction.

12. (canceled)

13. The drive unit of claim 1, wherein the drive unit comprises a housing and a key positioned between a drive interface element and the housing, wherein the key is secured to the housing and is configured to engage the respective drive interface element such that the drive interface element can be displaced relative to the key in a direction along the longitudinal axis of the drive interface element, and wherein the drive interface element comprises a slot, the slot being parallel to the longitudinal axis of the drive interface element, and the key is configured to slide within the slot when the drive interface element is displaced along its longitudinal axis.

14. (canceled)

15. A robotic surgical instrument configured to engage a surgical robot arm, the instrument comprising:
a plurality of instrument interface elements, each instrument interface element having a longitudinal axis,
the instrument being configured such that, when the instrument engages the surgical robot arm, the longitudinal axis of each instrument interface element is aligned with a longitudinal axis of a respective drive interface element in the surgical robot arm and each instrument interface element is configured such that a displacement of the respective drive interface element along its longitudinal axis in a first direction causes a displacement of the instrument interface element along its longitudinal axis in the first direction.

16. The robotic surgical instrument of claim 15, wherein the instrument is configured such that, when the instrument engages the surgical robot arm, the longitudinal axis of each instrument interface element is colinear with the longitudinal axis of the respective drive interface element in the surgical robot arm.

17. The robotic surgical instrument of claim 15, wherein the instrument is configured such that, when the robotic surgical instrument engages the surgical robot arm, the plurality of instrument interface elements are not secured to the respective drive interface elements.

18. The robotic surgical instrument of claim 15, wherein the instrument is configured such that, when the instrument engages the surgical robot arm, each instrument interface element is configured to be engaged by a pad of a load cell unit of the surgical robotic arm such that a displacement of the respective pad in the first direction causes a displacement of the instrument interface element along its longitudinal axis in the first direction, and wherein the instrument is configured such that, when the instrument engages the surgical robot arm, the instrument interface elements are not secured to the respective pads of the load cell unit.

19. (canceled)

20. The robotic surgical instrument of claim 15, the instrument comprising:
a shaft having a longitudinal axis extending between a proximal end and a distal end, and
an articulation and an end effector disposed at the distal end of the shaft, the articulation being configured to articulate the end effector,
wherein the plurality of instrument interface elements are disposed at the proximal end of the shaft and the longitudinal axis of each of the instrument interface elements is parallel to the longitudinal axis of the shaft.

21. The robotic surgical instrument of claim 20, wherein the first direction is from the proximal end to the distal end of the shaft.

22. The robotic surgical instrument of claim 20, wherein the instrument is configured such that, when the instrument engages the surgical robot arm, each instrument interface is secured to a driving element, such that the instrument is configured to translate drive from each of the driving elements into articulation of the end effector.

23. (canceled)

24. The robotic surgical instrument of claim 15, wherein each instrument interface element is configured such that the displacement of the respective drive interface element along its longitudinal axis in a second direction opposite to the first direction does not cause a displacement of the instrument interface element.

25. The robotic surgical instrument of claim 15, wherein each instrument interface element is configured such that the displacement of the respective drive interface element along its longitudinal axis in a second direction opposite to the first direction causes a displacement of the instrument interface element along its longitudinal axis in the second direction.

26. (canceled)
